(19) United States
(12) Patent Application Publication
(10) Pub. No.: US 2011/0281333 A1
(43) Pub. Date: Nov. 17, 2011

(54) METHANE PRODUCTION FROM SINGLE-CELL ORGANISMS

(76) Inventors: Paul W. Brown, State College, PA (US); Wendy E. Brown, West Sacramento, CA (US)

(21) Appl. No.: 13/107,196

(22) Filed: May 13, 2011

Related U.S. Application Data
(60) Provisional application No. 61/334,825, filed on May 14, 2010.

(51) Int. Cl. C12N 1/20

(52) U.S. Cl. 435/252.1

(57) ABSTRACT

The present invention relates to a method for enhancing the growth of single-cell organisms, such as methanogens. The growth of the single cell organisms includes consuming carbon dioxide to produce methane. The method can include providing a porous solid having an internal surface with a surface charge density, adhering the single-cell organism to the internal surface of the porous solid, populating the internal surface with the single-celled organism at least to confluence, introducing to the single-cell organism essential macronutrients consumed in the production of methane, and controlling the temperature conditions and pH conditions to allow the single-cell organism to produce methane.

### FIELD OF THE INVENTION

The present invention relates to single-cell organisms for producing methane, such as, in particular, methanogens, and methods for enhancing the growth and adherence of said organisms on a surface. Furthermore, the present invention relates to enhancing the production of methane by the single-cell organisms.

### BACKGROUND OF THE INVENTION

Organisms can be characterized as eukaryotes or prokaryotes. The distinction between these two terms is that eukaryotes isolate DNA within a nuclear membrane and prokaryotes do not. Single celled prokaryotes may be further characterized as archaea or bacteria. However, the archaea may be referred to as archeobacteria. Further, it is not uncommon for archaea to generally be referred to as bacteria. Archaea are diverse and may be further characterized based on various features, such as, but not limited to, the substrates on which they act, their habitat, their shapes, and the like. Methanogens are archaea that produce methane as a by-product of their growth. Other examples of archaea include the following: acidophiles (acid-loving), halophiles (salt-loving) which require highly saline environments, and thermophiles which can be characterized as being extremely thermophilic, moderately thermophilic, or mesophilic (all prefer heat, e.g., are heat-loving, but can have different optimal growth temperatures). Particular methanogens prefer each of, or a combination of, these conditions. For example, for some methanogens, temperatures of about 80° C. are preferable and others live in environments where the temperature exceeds that of the normal boiling temperature of water. Still, for other methanogens, cold temperatures are preferable. Thus, based on the thermal characteristics of a CO₂-containing environment, different strains of methanogens can be preferable for the production of methane.

Methane-generating bacteria are known as methanobacteriacea. The present invention relates to single cell organisms that produce methane as a result of their growth and does not depend on, nor is limited by, whether they are classified as bacterial or archeal. As used herein, such an organism will be referred to by the term “methanogen”.

As of 2003, there were identified 26 strains of methanogens belonging to 13 genera identified [Wright and Pimm, J. Microbiol. Methods, 337-49 (2003)]. Non-limiting examples of the strains of methanogens that have been identified include, but are not limited to, the following:
Methanobacterium bryantii,
Methanobacterium formicum,
Methanobrevibacter arboriphilicus,
Methanobrevibacter gottschalkii,
Methanobrevibacter ruminantium,
Methanobrevibacter smithii,
Methanocalculus chunghsingensis,
Methanococcoides burtonii,
Methanococcus aeolicus,
Methanococcus deltae,
Methanococcus jannaschii,
Methanococcus maripaludis,
Methanococcus vannielii,
Methanocorpusculum labreanum,
Methanoculleus bourgensis (Methanogenium olentangyi & Methanogenium bourgense),
Methanoculleus marisnigri,
Methanofollis liminatans,
Methanogenium cariaci,
Methanogenium frigidum,
Methanogenium organophilum,
Methanogenium wolfei,
Methanomicrobium mobile,
Methanopyrus kandleri,
Methanoregula boonei,
Methanosaeta concilii,
Methanosaeta thermophila,
Methanosarcina acetivorans,
Methanosarcina barkeri,
Methanosarcina mazei,
Methanosphaera stadtmanae,
Methanospirillium hungatei,
Methanothermobacter defluvii (Methanobacterium defluvii),
Methanothermobacter thermautotrophicus (Methanobacterium thermoautotrophicum),
Methanothermobacter thermoflexus (Methanobacterium thermoflexum),
Methanothermobacter wolfei (Methanobacterium wolfei), and
Methanothrix sochngenii.

The names above-mentioned are formulated based on various factors. For example, the name may acknowledge a prominent bacteriologist, it may be descriptive of the environment preferred by the methanogen or it may be descriptive of the morphology of the methanogen. “Methano” can be abbreviated as M. Thus, for example, the name M. thermoautotrophicum identifies the organism as a heat-loving, autotropic methanogen. If a species of methanogen is autotropic it has the ability to synthesize the carbon-based materials it needs by using carbon dioxide (CO₂) as its only carbon source. It is expected in the art that other methanogens will be identified, cultured and characterized in the future.

In general, methanogens are highly diverse. However, various methanogens have common traits. For example, the name “methanogens” is indicative of its metabolic activity in that they consume hydrogen (or hydrogen-containing organic compounds) to reduce carbon dioxide (CO₂) and to produce methane gas. The methanogens that use CO₂ as their only carbon source are referred to as “autotropes” or “lithotropes”. The energy needed to support their metabolic functions is produced by CO₂-reducing reactions. The following is an example of a CO₂-reducing reaction.

4H₂+H⁺+HCO₃⁻→CH₄+3H₂O+energy

In this equation, the CO₂ is dissolved in water and the dissociate is expressed as H⁺+HCO₃⁻. Some methanogens also use other sources of carbon, such as organic compounds. Further, some methanogens use organic compounds as a substitute for CO₂. Methanogens extract the energy they need through enzymatically-mediated reactions between a carbon source and hydrogen. The carbon source and hydrogen are referred to as “substrates” on which methanogens act. As used herein and the claims, the term “substrate” describes a molecule undergoing a reaction with an enzyme.

Methanogens can have at least one of the following properties and characteristics: (i) obligate anaerobes (require an environment with a low partial pressure of oxygen); (ii) use hydrogen as the exclusive source of energy for reducing oxidized forms of carbon (e.g,. CO₂); (iii) require a source of nitrogen to produce amino acids; (iv) require a source of sulfur to produce amino acids; and (v) require a source of phosphate to produce adenosine triphosphate. Methanogens can grow in natural environments, such as swamps and waterlogged wood. In these environments, the conditions necessary for methanogen growth are inherent. Other suitable environments include, but are not limited to, landfills and digesters. It is known in the art that various biodigesters can produce combustible concentrations of methane. Further, although conditions inherent for methanogen growth exist in various bio-reactor systems, it is believed that optimizing these systems to maximize the rate of methane produced has not been realized with regard to providing the methanogens optimal surfaces on which to populate. Further, it is believed that biodigester systems have been designed to operate at elevated pressures, to accept CO₂ produced by the combustion of hydrocarbons or to accept hydrogen generated externally to a biodigester system and then introduce to it. It is also believed that biodigesters do not provide gallery surfaces designed to be populated to confluence by methanogens. Thus, the rate of methane production in biodigester systems is not optimal and there is room for improvement. Therefore, it is an object of the present invention to both provide highly porous solids to the methanogens and to tailor the characteristics of those surfaces to facilitate the adherence methogens to them.

The growth of methanogens is not inherent for locations suitable for the underground storage of CO₂ that did not previously contain hydrocarbons and the trace or minor nutrients required for their growth. In one embodiment, underground storage of CO₂ includes drilling deep wells in order to inject CO₂ into porous rock formations that are overlain by low permeability formations, such as shales or claystones. This includes injection into sedimentary basins and large, horizontal aquifers. In another embodiment, CO₂ is injected into formations depleted of natural gas.

The conditions produced by injecting CO₂ below ground or storing CO₂ above ground do not produce an environment suitable for the growth of methanogens. Thus, it is another object of the present invention to provide for processing of CO₂-containing gases by creating conditions wherein a methanogen can act on such gases. A further object of the present invention is to improve or maximize the conversion of CO₂ to methane.

Still, another object of the present invention is to provide to the single-cell organism a porous solid. The single-cell organism will preferentially invade the porous solid and reside on an internal surface of the porous solid.

In yet another object, the present invention provides to the single-cell organism trace nutrients needed to permit the single-cell organism to produce methane.

### SUMMARY OF THE INVENTION

The present invention provides a method of enhancing the growth of a methane-producing single-cell organism. The method includes providing a porous solid having an internal surface with a surface charge density, adhering the single-cell organism to the internal surface of the porous solid, populating the internal surface with the single-celled organism at least to confluence, introducing to the single-cell organism essential macronutrients consumed in the production of methane, and controlling the temperature conditions and pH conditions to allow the single-cell organism to produce methane.

### DETAILED DESCRIPTION OF THE INVENTION

The present invention provides a method for enhancing or optimizing the growth of single-cell organisms and their adherence on a substrate. In one embodiment, the single-cell organism can be a methanogen. In alternate embodiments, the methanogen can include a single strain or multiple strains. In this embodiment, the present invention provides a method for enhancing or optimizing the production of methane by the methanogen.

Without intending to be bound by any particular theory, it is believed that appropriate or optimum conditions can be selected to enhance or optimize the growth of the single-cell organisms, the adherence of the single-cell organisms on a surface, and the rate at which these single-cell organisms, such as methanogens, produce methane. In alternate embodiments, the appropriate or optimum conditions for growth and methane production are selected by specifying and controlling at least one of temperature, pressure and pH. In other embodiments, the adherence of single-cell organisms on a surface can be facilitated by providing substrates or surfaces having appropriate characteristics, such as a high specific surface area, porosity, and an electrical charge. Moreover, nutrients required for growth and methane production can be delivered to the single-cell organisms by various mechanisms including, but not limited to, incorporating the nutrients into the substratum (e.g., pores or voids) to which the single-cell organisms adhere.

The term “growth” as used herein refers to the reproduction of bacteria-like organisms and does not require an increase in their physical size. The term “galleries” as used herein refers to interconnected pore or void spaces.

For ease of description, the present invention is described herein referring to methanogens. However, it is understood that the present invention includes and encompasses single cell organisms other than methanogens.

In general, methanogens require a carbon source and a hydrogen source for their growth and methane production. These sources can be referred to as major nutrients or macronutrients. The carbon source can be selected from a wide variety known in the art, such as but not limited to elemental carbon, CO₂, CO, carbon-containing organic compounds, and mixtures thereof. The hydrogen source can be selected from a wide variety known in the art, such as but not limited to elemental hydrogen, a hydrogen-containing organic compound, and mixtures thereof. In one embodiment, the carbon source is CO₂ In another embodiment, the hydrogen source is hydrogen extracted from hydrocarbon. The growth of the methanogen consumes carbon and hydrogen to produce methane.

In another aspect of the invention, an artificial environment is created or a natural environment is modified to facilitate methanogen growth. Methanogen growth can be employed as a means for processing CO₂ in any natural or artificial environment in which the pressure of CO₂ gas substantially exceeds atmospheric and, more particularly, at CO₂ pressures encountered in storage facilities. In one embodiment, the present invention provides for the processing of CO₂-containing gases by creating conditions in CO₂ storage chambers that are favorable for the growth of single-cell organisms. In this embodiment, the single-cell organisms consume the CO₂ to produce methane.

Still, another aspect of the invention is to provide suitable surfaces to enhance or optimize the adherence of methanogens thereon. In one embodiment, porous solids having a high surface to volume ratio (e.g. high specific surface area) are provided for methanogens to adhere onto. Natural formations may not contain a high specific surface area solid and therefore, these solids can be provided in order to promote or enhance methanogen growth.

A further aspect of the invention is to provide minor or trace nutrients, such as, but not limited to, a source of ammonia, a source of sulfur, a source of phosphate, and mixtures thereof. These minor or trace nutrients may be referred to as micronutrients. These nutrients may be incorporated within the porous solid or may be added to an aqueous solution in contact with the methanogens.

Furthermore, the method of the present invention includes providing chemical conditions which are conducive to methanogen growth, such as, for example, an appropriate pH. The injection of CO₂ into some natural formations, such as those where exposure to basic aluminosilicate minerals occurs, can produce bicarbonate brines having pH values suitable for the growth of methanogens. For other natural formations, it may be necessary to introduce buffer materials and water to create an environment conducive to methanogen growth. For example, to convert CO₂ stored in a former natural gas field to methane, an aqueous solution or formulation is injected therein to form cellular concrete in situ to provide an optimal environment for methanogen growth. As used herein and the claims, the term “cellular concrete” means that the concrete contains a large number of intentionally formed voids. This environment consists of galleries having walls populated by the methanogens. The in situ formation of cellular concrete can achieve at least one of the following objectives: provides a high surface area solid on which methanogens preferably adhere by providing surface-chemical characteristics appropriate for methanogen adhesion; tailoring the sizes and connectivities of the pores in the solid to optimize the surface area for methanogen interaction, and incorporating within the solid, trace or minor nutrients essential to the growth of methanogens.

A typical cellular concrete mixture includes Portland cement, water, and particles of aluminum (Al) metal. Optionally, the cellular concrete mixture can include mineral aggregates. In one embodiment, the Portland cement can include the presence of iron hydroxide, silica and combinations thereof. The cement undergoes hydration reactions and produces high pH conditions that cause Al metal powder to convert to Al₂O₃. In this process, hydrogen gas is generated which forms bubbles (e.g., gaseous porogens) within the mixture. The cement hardens around these bubbles and creates a void-filled, very low density, high porosity solid. It is significant that this process will produce a porous solid that contains galleries having high specific surface areas to which methanogens can adhere to permit the solid to be populated by large numbers of methanogens or methanogens along with other microorganisms that participate in the eventual formation/production of methane. The introduction of CO₂ to these galleries converts the hydrating cement to a calcium carbonate-like solid.

In an embodiment, the porous cement-forming mixture can be designed and constituted to contain one or more degradable, water-soluble, inorganic or organic materials in fibrous form such as starch, wood or paper fibers. These materials can be added to increase the internal surface area of the substrate or solid and to provide paths between the voids to permit the methanogens access to interior voids of the substrate or solid to facilitate elevated methanogen populations. The physical dimensions of these fibrous additions are selected based on the physical dimensions preferred by colonies of methanogens and may depend on the strain of methanogen that is preferable for a given reaction temperature or temperature range. The addition of these materials can result in at least one of the following: modifying the porosity of the porous solid and, providing a source of micronutrients and/or macronutrients. Optionally, other suitable inorganic or organic materials that can serve as substrates for the methanogens may also be added to facilitate the establishment of colonies.

In another embodiment for producing an environment conducive to the growth of methanogens, a buffering agent including cement kiln dust or other base-forming material, such as but not limited to sodium carbonate, sodium bicarbonate, alkali phosphate and mixtures thereof, can be injected into a natural formation used as a CO₂ storage chamber. The term “base” in this instance refers to the chemical compound related meaning of base, e.g., acid-base. Kiln dust is a by-product of cement production. It is basic and is rich in the oxides of Na, K and Ca. The kiln dust addition provides a mechanism for elevating the pH, thereby providing an opportunity for reactions to occur to form calcium silicate hydrate by reactions with native minerals, and to provide a buffering capacity associated with the formation of NaHCO₃ when CO₂ is introduced.

In the embodiments wherein cellular concrete is produced or kiln dust is introduced, the cellular concrete or the kiln dust reacts with CO₂. The cellular concrete reacts to decompose the normal hydrated cement-binding phases with the formation of CaCO₃ and silica gel. It is believed that a benefit of forming calcium silicate hydrate on existing mineral surfaces is that it carbonates when exposed to CO₂ and the resulting calcium carbonate-like mineral provides a preferred surface for the growth of methanogens. The kiln dust reacts to produce alkali bicarbonate solution with a pH that is appropriate for the growth of methanogens. Without intending to be bound by any particular theory, it is believed that binding of CO₂ with basic cementing phases, or with Ca(OH)₂ in particular, may result in more CO₂ being generated in producing cement or CaO than would be sequestered by these compounds.

Methanogen growth requires certain nutrients which can include, but are not limited to, the following:
Sources of carbon: CO₂, acetate, alcohol, formate, CO, NaHCO₃, and mixtures thereof;
Sources of hydrogen: H₂, hydrocarbons (acetate, formate), and mixtures thereof;
Sources of nitrogen: ammonia, amines, and mixtures thereof;
Sources of sulfur: H₂S, cysteine, and mixtures thereof;
KH₂PO₄ as a buffer and source of phosphate for ATP;
K₂HPO₄.3H₂O as a buffer and source of phosphate for ATP;
NH₄Cl as a nitrogen source for amino acids;
MgCl₂ for Mg as a component of some enzymes;
Na₂S.9H₂O as S is an essential component of some amino acids; and
Trace minerals as sources of Ni²⁺, Fe²⁺, Mn²⁺, Co²⁺, Zn²⁺, Ca²⁺, HBO₃⁻, MoO₄⁻.

Each of the aforementioned nutrients, and combinations thereof, can be provided by various methods and techniques. For example, the degradable organic fibers can be soaked in solutions containing the nutrients, the nutrients can be provided in the mixing water used to make the cellular concrete or the nutrients can be added with the cement as solids. Thus, it is an aspect of the present invention to provide the addition of trace minerals required for methanogen growth to storage chambers. In one embodiment, the addition of the trace minerals is provided as a result of the in situ formation of galleries produced using carbonated Portland cement.

In general, the temperature increases by about 25° C. for every kilometer of depth below the earth surface. Thus, to produce methane by growth of a methanogen or methanogens, the strain or strains are selected from those which grow optimally at the local temperature. For storage facilities which cover a range of depths, it is anticipated that different strains may be optimal at different depths. For example, thermophilic methanogen strain CB12, DSM 3664 can be grown at temperatures between about 30 and 70° C. while showing an optimum growth rate at or near 54° C. However, it is also recognized that methanogens can be trained to grow under conditions that would not be considered native to them.

In one embodiment of the present invention, the temperature conditions for growth of the methanogen includes a temperature of below room temperature to less than or equal to 100° C. It is known in the art that room temperature is from about 20° C. to about 25° C.

The CO₂-containing environment need not be underground and this is not a requirement for employing the growth of methanogens as a means for generating methane.

Depending on the strain, methanogens can exhibit optimal growth over differing ranges of pH. However, in an embodiment, the pH is at least 5 or 9 or less. In another embodiment, the pH is in the range of from 5 to 9. For example, the thermophilic methanogen strain CB12, DSM 3664 when grown at 60° C., has been known to show the growth over a range of pH values from 6 to 10 with a maximum growth rate at about 7.8 to 8. In one embodiment, conditions can be selected or controlled to produce pH conditions in CO₂ storage chambers which result in improved or optimal methanogen growth. In this embodiment, kiln dusts, other bases or buffering agents, such as but not limited to, sodium carbonate, sodium bicarbonate, alkali phosphate, and mixtures thereof, can be added to achieve the desired pH conditions.

It is generally accepted that methanogens can grow at normal atmospheric pressure. It has been shown that the methanogen M jannaschii will grow about 3 times faster at 92° C. when the pressure of CO₂ is increased from 7.8 atm to 250 atm. Growth under elevated pressure conditions associated with CO₂ sequestration has been demonstrated. Thus, the present invention includes providing or creating pressure conditions in CO₂ storage chambers which result in improved or optimal methanogen growth. In one embodiment, the elevated pressure conditions are provided by artificial means. The pressure can include those pressures that are known in the art for designing CO₂ storage wells.

Improved or optimal methanogen growth can be defined in terms of one or more of pressure, temperature, pH, substrates, the sources of carbon, the sources of hydrogen, and the availability of trace nutrients. Further, the optima for these can vary depending on the characteristics or strain or type of methanogen. In alternate embodiments, more than one of the pressure, temperature, pH, and addition of trace nutrients are simultaneously varied to achieve optimal results. For example, methanogen growth rates have been shown to increase by 100× with the addition of increasing amounts of NH₄Cl up to 20 mM/liter of the growth media.

Methanogens may be Gram positive (+) or Gram negative (−). Gram-positive methanogens usually have a thick mesh-like cell wall that stains purple while Gram-negative methanogens usually have a thinner wall that stains pink Gram-negative methanogens also have a lipid-containing outer membrane that is separated from the cell wall. The cell wall of an organism that stains Gram positive contains teichoic acids. A portion of these acids are associated with lipids and form lipoteichoic acids. These compounds can create a negatively charged or zwitterionic network that extends from the cell membrane to its surface. This confers the Gram positive cell wall a negative charge. Gram negative cell walls have outward facing membranes composed of phospholipids and lipopolysaccharides that are relatively highly negatively charged. Based on these characteristics, some surfaces are more attractive for attachment of methanogens than other surfaces. Thus, the hydrophilic and electrostatic properties of the walls of the galleries in the environment to which a methanogen is introduced can influence the adhesion of that methanogen to the wall. It is known in the art that bacteria growing in natural ecosystems are surrounded by glycocalyx and commonly grow in glycocalyx enclosed colonies adherent to surfaces. Further, it has been accepted that macromolecules bind to surfaces via ion pair. Without being bound by any particular theory, it is believed that the attachment of a methanogen to a solid surface can be facilitated by customizing or specifying the chemistry of that surface and tailoring or specifying the chemistry of the gallery walls to faciliate the attachment or adherence of methanogens. Such attachment or adherences is needed in order for the methanogens to grow.

Mineral surfaces when exposed to water or aqueous solutions can take on an electrical charge. Depending on the pH of the solution and the ions dissolved in it, the charge may be positive or negative. There is generally a pH value at which the mineral surface is neither positively nor negatively charged and this is referred to as the point of zero charge (PZC). The points of zero charge can vary for known various minerals. For solution pH values above its PZC, the surface of a solid in contact with it will be negatively charged and for values below its PZC, the surface will be positively charged. In addition, the farther from the PZC the greater the charge. Thus, if it is desired that a methanogen adhere to a solid surface, electrostatic considerations indicate that the negatively charged cell wall or the negatively charged glycocalxy will more readily associate with a surface that is not strongly negatively charged. Therefore, silica surfaces (PZC pH 2.3) may not be preferred host surfaces for methanogens. Consequently, selecting a natural formation containing mineral surfaces which have PZC's above or in the range of the pH values for optimal methanogen growth can facilitate the conversion of CO₂ to methane. For example, CaCO₃ is reported to have a PZC in the pH range of from 8 to 10.8; which is in the pH range or above the pH that is optimal for the growth of methanogens. Thus, electrostatic repulsion between a methanogen and a limestone-like surface is much lower than that between a methanogen and a silica-like (e.g. quartz-like) surface. Creating porous limestone-like galleries or producing lime-stone like surfaces by reacting active silicates with Ca(OH)₂ to produce and then carbonate calcium silicate hydrate, can facilitate methanogen adhesion.

Further, methanogens can adhere to negatively charged surfaces by attaining a separation distance associated with a secondary minimum. Theoretically, secondary minimum may be a feature of a plot of the potential energy of an interaction of a negatively charged object in solution (the methanogen) with a negatively charged suface as a function of the separation distance between the two. Thus, methanogen adhesion, even to negatively charged surfaces, is not precluded. The presence of polyvalent cations in solution (such as Ca⁺⁺ ion) can modify the electrostatics and promote conditions where adhesion is more favorable. Thus, soluble calcium salts, such as CaCl₂ or calcium acetate, may be introduced to the solution in contact with the galleries to faciliate adhesion. The subsequent precipitation of CaCO₃ on the surfaces of a subterranian formation in association with the injection of CO₂ results in the beneficial effect of favorably influencing the PZC of the surface of the formation.

In accordance with the present invention, methanogen adhesion can be facilitated by selection of surfaces which have appropriate PZC's or by the modification of the chemistry of the aqueous solution in contact with those surfaces or by the modification of the surfaces themselves. Modification can be achieved either by treatment of an existing surface, such as but not limited to, flushing an underground storage facility with solution having a high pH. Association of negatively charged surfaces with an NaOH-containing, Ca(OH)₂-containing or KOH-containing solution can result in monovalent or divalent cations associating with that surface, and in turn, can elevate the pH of its PZC. As described above, subsequent exposure to CO₂ can produce either a bicarbonate solution for Na and K or can precipitate a divalent carbonate such as Ca. These results may further condition the surfaces to facilitate methanogen association with them. In one embodiment, preparation of an optimal surface can be associated with (1) incorporating certain nutrients into a porous solid, (2) providing the solid with an interconnected pore structure that can provide a high specific surface area (high porosity either per unit weight or until bulk volume of the solid), and (3) selecting a solid with a PZC appropriate to facilitate methanogen adhesion or conditioning that surface to achieve an appropriate PZC. Such conditioning can be achieved by soluble polycationic compounds because these compounds can interact with negatively charged mineral surfaces, thereby producing surfaces having net positive charges. It is anticipated herein that these concepts may be applied to populating galleries with methanogens in order to produce methane from CO₂.

The present invention can include one or more of the following features and advantages.

The use of CO₂ that has been captured and concentrated in order to reduce green house gas emissions as a substrate to support the growth of methanogens (and other CO₂ consuming microorganisms) as a means of producing methane.

An artificial, completely inorganic environment (other than in culture) to grow methanogens.

The use of an artificially induced elevated pressure environment of CO₂ storage chambers (including but not limited to, above ground chambers, or bioreactors) to enhance the growth rates of methanogens.

The use of specific elevated temperatures (as are produced, for example, in deep subterranean environments) to provide conditions to improve or optimize the growth rates of methanogens.

The use of waste heat generated by power plants to provide the temperature conditions optimal to the growth of methanogens.

Specifying/customizing the solid surfaces to which methanogens will attach to optimize the rate of methane production.

Adding a source of NH₄ and a source of sulfur, such as NaS, phosphate or other trace mineral ingredients to natural, subterranean environments or to artificially produced galleries to provide the trace nutrients needed for methanogen growth.

Coupling methane production by methanogens with hydrogen and oxygen production by electrolysis of water.

The use of alkali hydroxide additions coupled with phosphate buffer additions to subterranean CO₂ storage chambers or above ground storage chambers to produce bi-carbonate solutions having pH values optimal for the growth of methanogens.

The use of cellular concrete to produce galleries for the methanogens.

The optimization of the total surface areas of these galleries to improve or maximize the rate of methane production per unit volume of the gallery. Such optimization is achieved via the selection of the size distribution and number of particles of aluminum metal and of the organic material, capable of providing a source of hydrogen to the methanogens, or a water soluble inorganic material, capable of dissolving in aqueous solution, added at the time of mixing. Criteria for optimization will be dependent on the strain of methanogen, the need for the wall of the porous solid to withstand the service environment without substantial collapse, and the need to ensure that liquid and gas flow is not limited by diffusion

The incorporation of degradable organic fibers into the cellular concrete.

The alteration the surface characteristics of the concrete itself to produce a surface to which methanogens will preferentially attach or adhere. Such alteration is dependent on the strain of methanogen and recognizes that the preferred sign and magnitude of the electrical change on the surface will depend on the polysaccharides that methanogen strain surrounds itself with.

The delivery of anesthetized methanogens (by nitrogen or by inert gases) to these preformed galleries of porous media or to native subterranean surfaces.

The introduction of a soluble calcium salt, such as CaCl₂ or calcium acetate, to the solution in contact with the galleries to faciliate adhesion of methanogens to the gallery walls.

The delivery of CO₂ produced by the combustion of fossil fuel to galleries or chambers populated by methanogens.

While the invention has been described in terms of various specific embodiments, those skilled in the art will recognize that the invention can be practiced with modifications within the spirit and scope of the appended claims.

We claim:

1. A method of enhancing the growth of a methane-producing single-cell organism, comprising:
providing a porous solid having an internal surface with a surface charge density;
adhering the single-cell organism to the internal surface of the porous solid;
populating the internal surface with the single-celled organism at least to confluence;
introducing to the single-cell organism essential macronutrients consumed in the production of methane; and
controlling the temperature conditions and pH conditions to allow the single-cell organism to produce methane.

2. The method of claim 1 where the macronutrients are selected from the group consisting of a carbon source, a hydrogen source, and combinations thereof.

3. The method of claim 2, wherein the hydrogen source is selected from the group consisting of hydrogen, a hydrogen-containing organic compound and mixtures thereof.

4. The method of claim 2, where the carbon source is carbon dioxide.

5. The method of claim 1, further comprising the introduction of micronutrients into the porous solid.

6. The method of claim 1, wherein the porous solid comprises Portland cement.

7. The method of claim 6, further comprising hydrating the Portland cement to produce galleries therein.

8. The method of claim 7, wherein the galleries are produced by mixing the Portland cement with aluminum metal powder to produce a gaseous porogen.

9. The method of claim 8, further comprising intermixing a source of water-soluble inorganic material with the Portland cement to obtain a result selected from the group consisting of modifying the porosity, providing a source of macronutrients and micronutrients, and combinations thereof.

10. The method of claim 9, wherein the source of water-soluble inorganic material is wood fiber.

11. The method of claim 6, further comprising adding to the Portland cement a material selected from the group consisting of iron hydroxide, silica and combinations thereof.

12. The method of claim 6, further comprising exposing the galleries to carbon dioxide.

13. The method of claim 1, wherein the pH conditions includes a pH of from 5 to 9.

14. The method of claim 1, wherein the pH conditions is controlled by adding a buffering agent.

15. The method of claim 14, wherein the buffering agent is selected from the group consisting of kiln dust, sodium carbonate, sodium bicarbonate, alkali phosphate, and combinations thereof.

17. The method of claim 1, wherein the single-cell organism is a methanogen.

18. The method of claim 1, wherein the temperature conditions includes a temperature from below room temperature to about 100° C.

19. The method of claim 1 where the pressure is elevated by an artificial means.
